(19) United States
(12) Patent Application Publication
(10) Pub. No.: US 2004/0193258 A1
(43) Pub. Date: Sep. 30, 2004

(54) Tubular structure

(76) Inventors: Julian G. Ellis, Nottingham (GB); Peter W. Butcher, Nottingham (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/483,887

(22) PCT Filed: Jul. 15, 2002
(86) PCT No.: PCT/GB02/03217

(30) Foreign Application Priority Data
Jul. 14, 2001 (GB) 0117199.0

(51) Int. Cl. A61F 2/06

(52) U.S. Cl. 623/1.49

(57) ABSTRACT

A stent having an elongate tubular body (11) formed from a seamless textile fabric having a predefined fabric structure, the tubular body (11) being biased by resilient support means into an erected tubular shape to define a conduit, the support means being in the form of one or more elongated resilient elements (31) anchored to said fabric structure so as to be constrained to extend circumferentially and/or longitudinally about the tubular body (11), the fabric structure including a series of anchorage loops (40) formed on a surface of the fabric, the anchorage (40) loops being spaced circumferentially around and/or longitudinally along the tubular body at predetermined locations to define one or ore predefined paths, said one or more elongate resilient elements (31) being arranged to pass through said anchorage loops (40) so as to be constrained to extend along said one or more predefined paths.

The present invention relates to a tubular structure for placement within a human or animal body.

Stents are tubular structures which are inserted into a lumen of a human or animal body, such as a blood vessel or air pipe, to maintain open free flow along the lumen of fluids such as gas and liquids and solids such as food.

Some stents are inserted into the lumen in a collapsed state and then allowed to expand, when in place, to open up the lumen.

A general aim of the present invention is to provide a stent which is easy to manufacture in a wide variety of diameters/tubular shapes.

According to one aspect of the present invention, there is provided a stent having an elongate tubular body formed from a seamless textile fabric having a predefined fabric structure, the tubular body being biased by resilient support means into an erected tubular shape to define a conduit, the support means being in the form of one or more elongated resilient elements anchored to said fabric structure, the fabric structure including a series of anchorage loops formed on a surface of the fabric, the anchorage loops being spaced circumferentially around and longitudinally along the tubular body at predetermined locations to define one or more predefined paths, said one or more elongate resilient elements being arranged to pass through said anchorage loops so as to be constrained to extend along said one or more predefined paths and thereby extend circumferentially and/or longitudinally about the tubular body.

The fabric structure may be woven or knitted.

Preferably said surface of the fabric forms the outer-surface of the tubular body.

Preferably, said one or more elongate resilient elements are laid-in said fabric structure.

The resilient elements may be formed from a suitable plastics material or metal.

According to another aspect of the present invention, there is provided a method of manufacturing a stent, the method including forming an elongate tubular body by converting yarn to produce a predetermined seamless tubular fabric structure, forming a series of anchorage loops on the surface of said fabric at predetermined locations to define one or more predefined paths, and anchoring to said fabric structure one or more elongate resilient elements by locating said elongate elements within said anchorage loops such that said one or more elongate resilient elements are constrained to extend circumferentially and/or longitudinally about said tubular body in order to erect said body into a tubular shape which defines a conduit.

Various aspects of the present invention are hereinafter described with reference to the accompanying drawings.

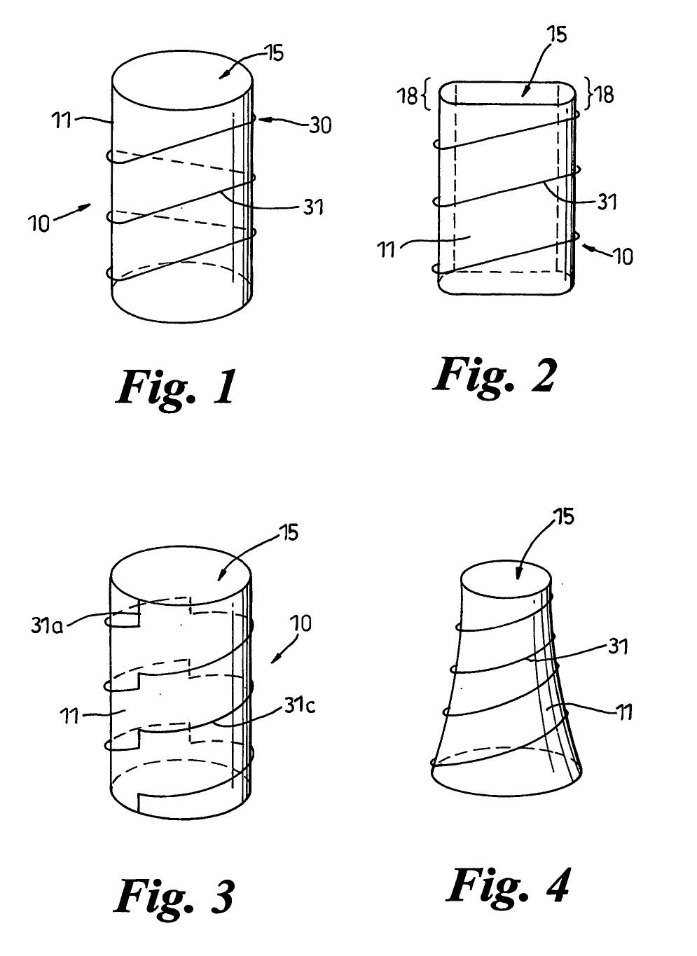

FIG. 1 is a schematic part perspective view of a stent according to a first embodiment of the present invention;

FIG. 2 is a schematic part perspective view of a second embodiment according to the present invention.

FIG. 3 is a schematic part perspective view of a third embodiment of the present invention;

FIG. 4 is a side view of a fourth embodiment according to the present invention;

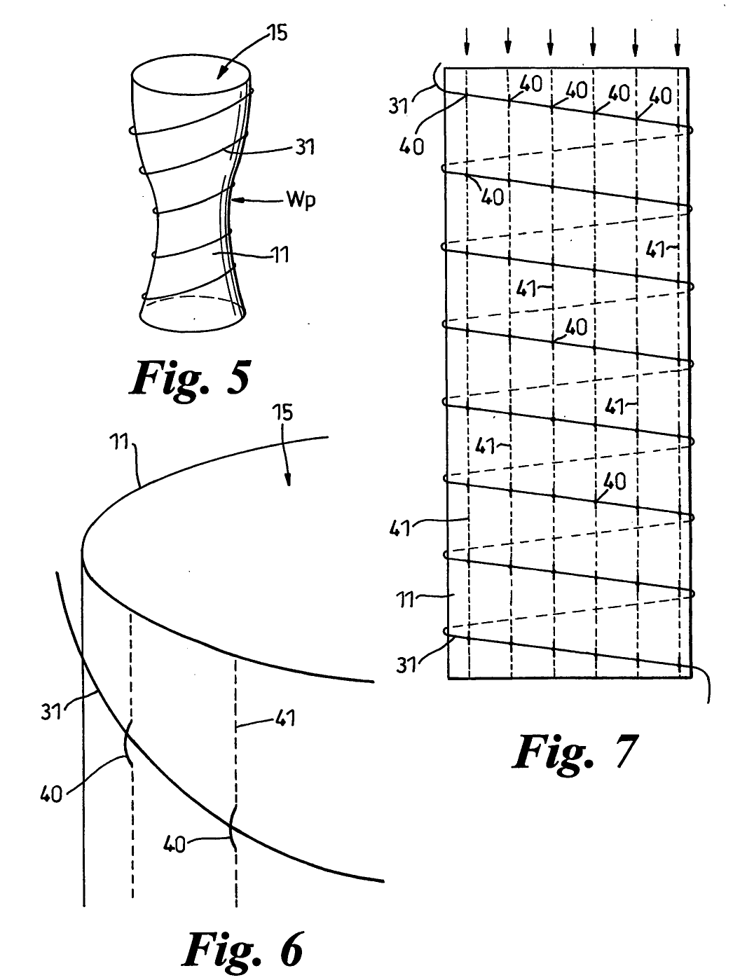

FIG. 5 is a schematic perspective view of a fifth embodiment;

FIG. 6 is an enlarged part perspective view of a sixth embodiment according to the present invention;

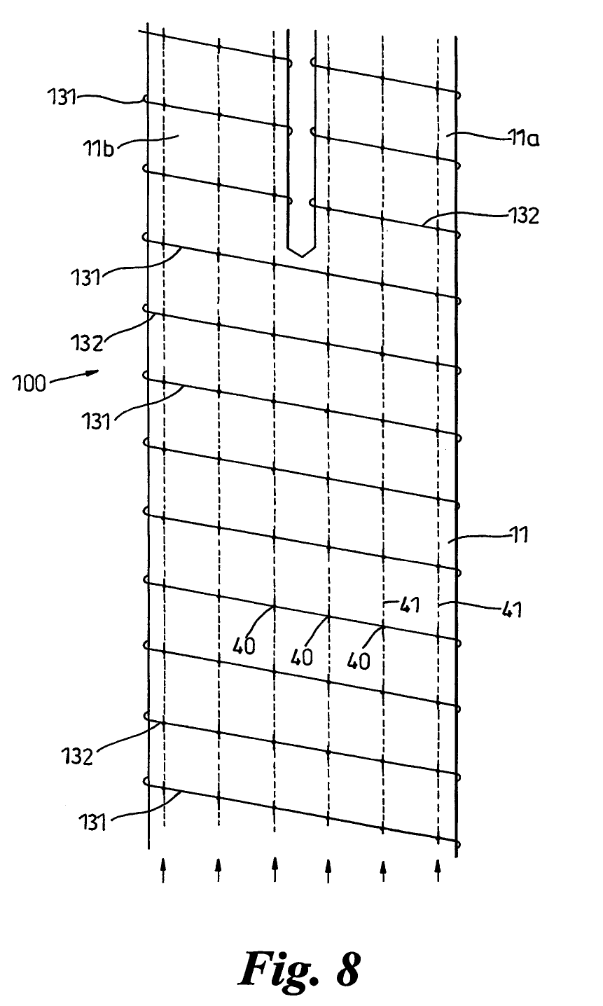

FIG. 7 is a side view of the sixth embodiment shown in its ‘as produced’ condition;

FIG. 8 is a side view of a seventh embodiment shown in its ‘as produced’ condition;

FIG. 9 is a side view of the third embodiment shown in its ‘as produced’ condition; and

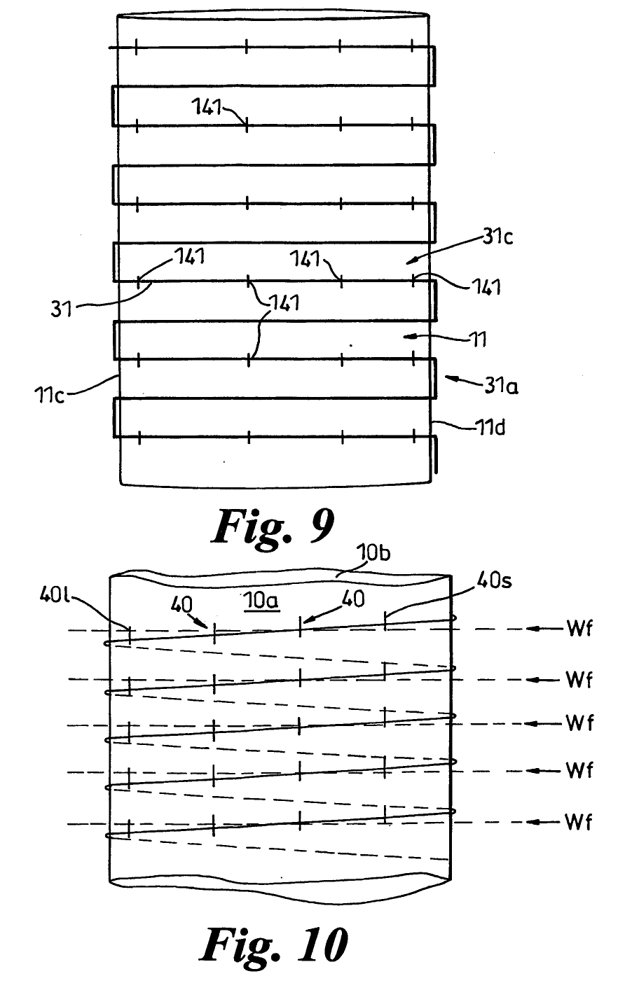

FIG. 10 is a side view of an eighth embodiment shown in its “in use” condition.

Referring initially to FIG. 1, there is shown a stent 10 according to a first embodiment of the present invention.

The stent 10 includes an elongate tubular body 11 formed from a seamless tubular textile fabric having a predefined fabric structure. The textile fabric may be a knitted or a woven fabric. If knitted, the fabric is preferably a weft knitted fabric which is produced either on a circular knitting machine or on a straight bed machine having two sets of needles.

In this specification the term ‘seamless’ tubular fabric is intended to mean that the fabric is formed into a tube by means of the knitted/woven structure and not by means of a seam joining opposed edges of a strip of fabric together.

Preferably the textile fabric is either knitted or woven so as to create tubular bodies 11 which may range in sizes between about 2 mm diameter and about 35 mm diameter.

The tubular body 11 is produced from textile yarns suitable for location within a lumen of a human or animal body. Preferably these yarn are not water-soluble. Suitable yarns are polyester or polyamide yarns.

The tubular body 11 is knitted or woven so as to be relatively flexible such that when removed from the knitting machine or loom it has little resistance to flexure along its length. In view of this, the tubular body 11 has little inherent resilient capability to assume an erected tubular shape which defines an open conduit 15.

The stent 10 of the present invention is therefore provided with resilient support means 30 which inter-act with the tubular body 11 to open the tubular body 11 to form an erected tubular shape and define an open conduit 15.

Preferably the resilient support means 30 is in the form of one or more resilient elongate elements 31 which extend circumferentially around and/or longitudinally along the tubular body 11. The elements 31 are attached to the tubular body 11 and so the tubular body 11 is resiliently urged diametrically outwardly to define an open conduit 15.

Preferably the elements 31 act to resiliently urge the tubular body 11 outwardly in a diametric direction so as to place the fabric of the tubular body 11 under tension, i.e. the fabric of the body 11 is stretched in the circumferential direction.

This is preferable as it means that the tubular shape adopted by the tubular body may be determined by the fabric structure. In this respect it is envisaged that during knitting or weaving, the tubular body 11 may be provided with one or more longitudinal zones 18 which are relatively stiff. (See FIG. 2). When the tubular body 11 is expanded diametrically by the resilient element(s), the relatively stiff zones 18 are more resistive to diametric expansion and so a tubular cross-sectional shape other than a circular cross-section may be achieved. For example, by the provision of a pair of diametrically opposed stiff zones 18 it is possible to achieve an ovoid cross-sectional shape.

In FIG. 1, a single resilient element 31 is shown which extends circumferentially about and longitudinally along the tubular body 11 in a spiral or helical path.

In FIG. 3, a third embodiment is illustrated wherein a single resilient element 31 extends in a step-wise manner circumferentially about and longitudinally along the tubular body 11. In this respect, in FIG. 3, the resilient element 31 has longitudinally extending portions 31a separated by circumferential portions 31c. The longitudinal portions 31a have an axial or longitudinal extent only and are preferably aligned with one another. Accordingly, on axial compression of the stent 10, the portions 31a are brought into axial contact with one another and thereafter resist further axial compression of the tubular body 11.

The circumferential portions 31c have a circumferential extent only and serve to resiliently expand the tubular body 11 diametrically.

It is envisaged that the resilient elements 31 may be formed from a suitable resilient metal wire or a suitable plastics filament.

In FIGS. 1,2 and 3, the tubular body 11 is illustrated when expanded as an elongate tubular shape of constant cross-sectional dimensions along its length.

It is envisaged, as illustrated in FIG. 4, that the cross-sectional dimensions along the length of the tubular shape defined by tubular body 11 may be varied so as to have a progressively increasing diameter from one end to the other. This may be achieved, for example, during the knitting process by changing the stitch structure and/or yarn tensions as the tubular body 11 is knitted from one end to the other. Also, it is envisaged that this technique could be used to provide the stent with a waisted portion Wp as illustrated schematically in FIG. 5.

It will be appreciated that by suitable choice of the knitting or weaving technique, the diametric size, and tubular shape of the stent may be tailored to fit the lumen of the human or animal body with which the stent is to be fitted.

In addition, since the tubular body 11 is knitted or woven to form a tube, it is possible to produce stents of a smaller diameter than has before been possible.

As seen in FIG. 8, it is also possible with the technique of the present invention to produce a stent 100 having a main body portion 11 from which extends a pair of auxiliary body portions 11a, 11b.

In the embodiment of FIG. 8, two resilient elements 131, 132 are provided. These each extend about the main body portion 11 with one element 131 extending about body portion 11b only and element 132 extending about body portion 11a only.

Attachment of the resilient element 31 to the tubular body 11 may be achieved in different ways.

As illustrated in FIGS. 6 and 7, the outer surface of tubular body is provided with a series of anchorage loops 40. The loops 40 are produced so as to be located at predetermined locations on the tubular body 11 so as to define one or more predefined paths for the elongate support elements 31 to follow. The loops 40 may be formed during the knitting or weaving process by the provision of floats at said predetermined locations. Alternatively, the loops 40 may be formed by sewing an anchorage thread 41 into the tubular body 11 at said predetermined locations, by, for example, embroidering techniques. Use of an embroidery-sewing machine enables the position of the loops 40 to be accurately made.

With this method of anchorage, the resilient element 31 can be inserted subsequent to weaving/knitting of tube 11. The element 31 may be preshaped, e.g. in a spiral or may be formed from a material having a shape memory such that after insertion the material may be heated to a predetermined temperature so as to assume its memorised shape, e.g. a spiral.

With this method of anchorage, it is possible to produce the embodiments as shown in FIGS. 1,2,4 to 8 having a spiral element 31. Alternatively, as illustrated in FIG. 9 the resilient element 31 may be incorporated into the tubular body 11 during the knitting or weaving process.

In FIG. 9 the resilient element 31 is shown as extending along one or other selvedge 11c. 11d for a predetermined number of courses before being transferred to the opposite selvedge. At the time of transfer from one selvedge to the other, the element 31 is anchored at desired locations on a selected course, preferably by a series of spaced loops 141 formed in the fabric during the knitting or weaving process, and so in effect floats along that course to produce a circumferential portion 31c. That portion of the element 31 extending along a selvedge in-between the selected courses defines longitudinal portions 31a.

In the embodiment shown, when the element 31 is passed from the selvedge 11c to selvedge 11d it passes over the upper outer surface of the fabric body 11 whereas when it is passed from the selvedge 11d to selvedge 11c it passes over the lower outer surface of the fabric body 11. Accordingly, after erection of the tubular body, adjacent portion 31c of the element 31 extend on opposite sides of the erected tubular body.

Since all the loops 141 are located during the same course for each insertion of the element 31, the portion 31c of element 31 is constrained to extend in a circumferential direction only.

With this mode of anchorage, the resilient element 31 is preferably formed from a shape memory material which in flexible during the time of knitting/weaving and which after knitting/weaving is heat treated to assume a predefined shape, e.g. spiral.

A further embodiment is illustrated in FIG. 10 wherein a technique similar to that described with respect to FIG. 9 is used for producing a stent 10 of the type having a spirally extending resilient member 31, e.g. as shown in FIGS. 1 and 2.

In the embodiment of FIG. 10, the resilient member 31 is inserted as a weft.

At the time of insertion of the resilient member 31 across the outer face of the top layer 10a of fabric, a series of wale extending loops 40 are being created by wale yarns, adjacent loops 40 being staggered in the warp direction and each loop 40 extending over a plurality of wefts.

The path of insertion of the resilient member 31 across the top layer 10a is indicated as W_{f} and it will be seen that, due to the staggered nature of the wale loops 40, this path is located adjacent to the start of a first loop 40s and adjacent to the finish of a last loop 401 and at the intermediate position with respect to the intervening loops 40.

During the next insertion, the resilient member 31 is inserted (shown in broken lines) across outer face of the lower layer 10b of fabric into wale loops 40 which are similarly staggered in the warp wise direction on the lower layer 10b.

Accordingly after weaving, it will be appreciated that the staggered loops 40 define a helical path. The resilient member 31 assumes a centralised position with respect to each loop 40 through which it passes and so, itself assumes a spiral.

In the embodiments described above, the resilient member 31 extends across the outer face of the tubular body 11. It is to be appreciated that the tubular body 11 may be turned inside out such that the resilient member 31 is located on the inside of the tubular body 11.

1. A stent having an elongate tubular body formed from a seamless textile fabric having a predefined fabric structure, the tubular body being biased by resilient support means into an erected tubular shape to define a conduit, the support means being in the form of one or more elongated resilient elements anchored to said fabric structure, the fabric structure including a series of anchorage loops formed on a surface of the fabric, the anchorage loops being spaced circumferentially around and longitudinally along the tubular body at predetermined locations to define one or more predefined paths, said one or more elongate resilient elements being arranged to pass through said anchorage loops so as to be constrained to extend along said one or more predefined paths and thereby extend circumferentially and/or longitudinally about the tubular body.

2. A stent according to claim 1 wherein said anchorage loops are formed as part of said fabric structure.

3. A stent according to claim 1 wherein said fabric structure is a knitted or woven structure.

4. A stent according to claim 3 wherein said one or more elongate resilient elements are laid-in said fabric structure.

5. A stent according to claim 4 wherein said fabric structure is a woven structure, said series of anchorage loops are defined by warp yarns and said one or more elongate resilient elements are defined by wefts laid-in said fabric structure so as to extend through said anchorage loops.

6. A stent according to claim 5 wherein said anchorage loops are arranged in circumferentially extending groups which are spaced longitudinally along the tubular body.

7. A stent according to claim 5 wherein the said anchorage loops are staggered from one another to define a helical path.

8. A stent according to claim 1 wherein said fabric structure defines a tubular body having varying diameter size along its length.

9. A stent according to claim 1 wherein the fabric structure defines a tubular body having a main tubular body portion from which extends a pair of auxiliary tubular body portions.

10. A method of manufacturing a stent, the method including forming an elongate tubular body by converting yarn to produce a predetermined seamless tubular fabric structure, forming a series of anchorage loops on a surface of said fabric at predetermined locations to define one or more predefined paths, and anchoring to said fabric structure one or more elongate resilient elements by locating said elongate elements within said anchorage loops such that said one or more elongate resilient elements are constrained to extend circumferentially and/or longitudinally about said tubular body in order to erect said body into a tubular shape which defines a conduit.

11. A method according to claim 10 wherein said body is formed by weaving, said anchorage loops are formed by warp yarns and said elongate resilient elements are inserted, as a weft, into said anchorage loops during the weaving process.
